(19) United States
(12) Patent Application Publication
(10) Pub. No.: US 2025/0012780 A1
(43) Pub. Date: Jan. 9, 2025

(54) Application of Organic Acids in Urine for Health Management

(71) Applicant: Yung-Chun Ou, Taipei City (TW)

(72) Inventor: Yung-Chun Ou, Taipei City (TW)

(21) Appl. No.: 18/357,161

(22) Filed: Jul. 24, 2023

(30) Foreign Application Priority Data
Jul. 4, 2023 (TW) 112124857

(51) Int. Cl. G01N 33/50; G16H 20/00

(52) U.S. Cl. CPC G01N 33/50; G16H 20/00; G01N 2560/00; G01N 2800/04; G01N 2800/085

(57) ABSTRACT

The present invention provides an application of organic acids in urine for health management, used for testing urine specimens of the human body by using a liquid chromatography tandem mass spectrometry to analyze the urine specimens to obtain multiple values of organic acids in the urine. The test values include ideal values and reference values; when the values are within the ideal value range, the health status is assessed as healthy; when the values are within the reference value range, the health status is assessed as sub-healthy; when the values are outside the ideal and reference value ranges, the health status is assessed as having a risk of developing diseases. Therefore, the application of organic acids in urine for health management according to the present invention enables the development of various health management and anti-aging plans, as well as prevention and treatment of chronic diseases.

### (a) TECHNICAL FIELD OF THE INVENTION

The present invention relates to an application of organic acids in urine for health management, and more particularly to a method for detecting organic acids in urine to assess the human body's health status.

### (b) DESCRIPTION OF THE PRIOR ART

With the progress of society, living standards have continuously improved, and people enjoy the convenience brought by civilization. However, the modern lifestyle has subjected the body to prolonged high-pressure environments, causing harm to health. Furthermore, as the living environment continues to change, many pollutants that harm us have emerged in our surroundings, impacting our bodies. In order to prevent further harm to our bodies, people can only minimize damage to the living environment as much as possible, and people can change daily dietary habits and exercise routines to achieve the goal of maintaining good health.

Additionally, with the increasing global aging population and declining birth rates, people are increasingly valuing quality of life and the path to wellness. Modern people fear the occurrence of disease, but ignore that disease development happens gradually. The process of developing disease in the body is continuous and uninterrupted. In the process of gradual decline in physical function, it is also our ability to grasp the golden time of returning to health or staying away from disease. People must know in the right place at the time whether the body function is changing or where it changes from. Therefore, a comprehensive health assessment in needed to gather information through a thorough functional evaluation, in order to further pursue improvement and nurturing.

In view of this, the inventor has invested a lot of research, development and effort, making breakthroughs and innovations, hoping to solve the current shortcomings with novel technical methods, not only bringing better products to the society, but also promoting industrial development at the same time.

### SUMMARY OF THE INVENTION

The main objective of the present invention is to provide an application of organic acid metabolites in urine for health management.

To achieve the above objective, the present invention provides an application of organic acids in urine for health management, used for the external testing of urine specimens from a human body, using a liquid chromatography tandem mass spectrometry to analyze the urine specimens to obtain multiple values of organic acids in the urine, and categorizing the organic acid values as the first indicator, second indicator, third indicator, and fourth indicator. First, the first indicator is selected from the group consisting of a fatty acid metabolism indicator, a carbohydrate metabolism indicator, and a mitochondrial energy generation indicator. Secondly, the second indicator is selected from the group consisting of a vitamin B complex indicator, a urea cycle indicator, an oxidative damage DNA and lipid indicator, an antioxidant indicator, and a liver detoxification indicator. Moreover, the third indicator is selected from the group consisting of a tryptophan metabolism indicator, a kynurenine metabolism indicator, a catecholamine metabolism indicator, and a catecholamine metabolism enzyme activity indicator. Further, the fourth indicator is selected from the group consisting of an intestinal digestion function indicator, a clostridia metabolism indicator, an intestinal bacterial overgrowth indicator, a fungal overgrowth indicator, and an oxalate metabolism indicator. In addition, each urine organic acid has an ideal value and a reference value, when the organic acid values fall within the ideal value range, the human's health status is evaluated as healthy; when the organic acid values fall within the reference value range, the human's health status is evaluated as sub-healthy; and when the organic acid values fall outside the range of both the ideal value and reference value, the human's health status is evaluated as at risk of disease.

In some embodiments, the fatty acid metabolism indicator comprises an adipate value, a suberate value and an ethlmalonate value; the carbohydrate metabolism indicator comprises a pyruvate value, a lactate value and a beta-hydroxybutyric acid value; and the mitochondrial energy generation indicator comprises a citrate value, a cis-aconitate value, a Isocitrate value, an alpha-ketoglutaric acid value, a succinate value, a fumarate value, a malate value, a hydroxymethylglutaric acid value.

In some embodiments, the ideal value for the adipate value is ≤5.15 μg/mg cr. and the reference value is ≤7.25 μg/mg cr., the ideal value for the suberate value is ≤4.58 μg/mg cr. and the reference value is ≤6.47 μg/mg cr., and the ideal value for the ethlmalonate value is ≤4.68 μg/mg cr. and the reference value is ≤6.12 μg/mg cr; wherein the ideal value for the pyruvate value is ≤2.06˜5.45 μg/mg cr. and the reference value is 0.23˜7.38 μg/mg cr.; the ideal value for the lactate value is 7.51˜24.1 μg/mg cr. and the reference value is 2.8˜32.8 μg/mg cr., and the ideal value for the beta-hydroxybutyric acid value is ≤2.27 μg/mg cr. and the reference value is ≤3.98 μg/mg cr.; wherein the ideal value for the citrate value is 241˜575 μg/mg cr. and the reference value is 63˜748 μg/mg cr., the ideal value for the cis-aconitate value is 49˜83.6 μg/mg cr. and the reference value is 29.5˜103 μg/mg cr., the ideal value for the isocitrate value is 58.3˜95.9 μg/mg cr. and the reference value is 38.8˜116 μg/mg cr., the ideal value for the alpha-ketoglutaric acid value is 5.5˜17.9 μg/mg cr. and the reference value is 0.9˜23.7 μg/mg cr., the ideal value for the succinate value is ≤10.8 μg/mg cr. and the reference value is ≤18.7 μg/mg cr., the ideal value for the fumarate value is ≤0.52 μg/mg cr. and the reference value is ≤0.79 μg/mg cr., the ideal value for the malate value is ≤1.34 μg/mg cr. and the reference value is ≤2.00 μg/mg cr., and the ideal value for hydroxymethylglutaric acid value is ≤5.96 μg/mg cr. and the reference value is ≤7.27 μg/mg cr.

In some embodiments, the vitamin B complex indicator comprises a α-ketoisovaleric acid value, a α-ketoisocaproic acid value, a α-keto-β-methylvaleric acid value, a xanthurenate value, a methylmalonic acid value, a N-Methylglutamic acid value and a β-Hydroxyisovaleric acid value; wherein the urea cycle indicator comprises an orotate value; wherein the oxidative damage DNA and lipid indicator comprises a deoxyguanosine (8-OHdG) value and a lipid peroxide (LPO) value; wherein the antioxidant indicator comprises an alpha-hydroxybutyric acid (AHBA) value and a pyroglutamate value; and wherein the liver detoxification indicator comprises a glucarate value.

In some embodiments, the ideal value for the α-ketoisovaleric acid value is ≤0.13 μg/mg cr. and the reference value is ≤0.2 μg/mg cr., the ideal value for the α-ketoisocaproic acid value is ≤0.63 μg/mg cr. and the reference value is ≤0.88 μg/mg cr., the ideal value for the α-keto-β-methylvaleric acid value is ≤1.37 μg/mg cr. and the reference value is ≤2.13 μg/mg cr., the ideal value for the xanthurenate value is ≤0.49 μg/mg cr. and the reference value is ≤0.63 μg/mg cr., the ideal value for the methylmalonic acid value is ≤2.27 μg/mg cr. and the reference value is ≤2.83 μg/mg cr., the ideal value for the N-Methylglutamic acid value is ≤0.28 μg/mg cr. and the reference value is ≤0.38 μg/mg cr., and the ideal value for the β-Hydroxyisovaleric acid value is ≤13.3 μg/mg cr. and the reference value is ≤17.0 μg/mg cr.; wherein the ideal value for the orotate value is ≤0.66 μg/mg cr. and the reference value is ≤0.90 μg/mg cr.; wherein the ideal value for the deoxyguanosine value is ≤4.78 μg/mg cr. and the reference value is ≤5.6 μg/mg cr., and the ideal value for the lipid peroxide value is ≤7.53 μg/mg cr. and the reference value is ≤9.8 μg/mg cr.; wherein the ideal value for the alpha-hydroxybutyric acid value is ≤6.12 μg/mg cr. and the reference value is ≤7.37 μg/mg cr., and the ideal value for the pyroglutamate value is 12˜22.1 μg/mg cr. and the reference value is 6.76˜27.3 μg/mg cr.; and wherein the ideal value for the glucarate value is ≤8.39 μg/mg cr. and the reference value is ≤11.0 μg/mg cr.

In some embodiments, the tryptophan metabolism indicator comprises a tryptophan value, a 5-hydroxyindoleacetic acid (5-HIAA) value, a kynurenine value, a kynurenate value, a picolinate value and an quinolinate value; wherein the kynurenine metabolism indicator comprises a IDO enzyme activity value and a KMO enzyme activity value; wherein the catecholamine metabolism indicator comprises a ferulic acid (HVA) value, a vanillin mandelic acid (VMA) value and a dihydroxyphenylacetic acid (DOPAC) value; and wherein the catecholamine metabolism enzyme activity indicator comprises a DBH enzyme activity value and a COMT enzyme activity value.

In some embodiments, the ideal value for the tryptophan value is 6.74˜17.1 μg/mg cr. and the reference value is 5.39˜23.4 μg/mg cr., the ideal value for 5-hydroxyindoleacetic acid value is 4.14˜8.10 μg/mg cr. and the reference value is 3.25˜10.1 μg/mg cr., the ideal value for the kynurenine value is 0.34˜1.11 μg/mg cr. and the reference value is 0.27˜1.44 μg/mg cr., the ideal value for the kynurenate value is ≤2.84 μg/mg cr. and the reference value is ≤4.86 μg/mg cr., the ideal value for the picolinate value is 0.12˜0.30 μg/mg cr. and the reference value is 0.072˜0.38 μg/mg cr., and the ideal value for the quinolinate value is ≤5.93 μg/mg cr. and the reference value is ≤6.96 μg/mg cr.; wherein the ideal value for the IDO enzyme activity value is 41.4˜90.4 μg/mg cr. and the reference value is 34.9˜104 μg/mg cr., and the ideal value for the KMO enzyme activity value is 1.38˜3.22 μg/mg cr. and the reference value is 0.85˜4.46 μg/mg cr.; wherein the ideal value for the ferulic acid value is 2.81˜9.32 μg/mg cr. and the reference value is 1.99˜10.4 μg/mg cr., the ideal value for the vanillin mandelic acid value is 2.89˜5.33 μg/mg cr. and the reference value is 1.97˜5.93 μg/mg cr., and the ideal value for the dihydroxyphenylacetic acid value is 0.72˜2.14 μg/mg cr. and the reference value is 0.54˜2.58 μg/mg cr.; wherein the ideal value for the DBH enzyme activity value is 0.87˜2.11 μg/mg cr. and the reference value is 0.61˜2.84 μg/mg cr., and the ideal value for the COMT enzyme activity value is 2.75˜6.28 μg/mg cr. and the reference value is 1.48˜7.86 μg/mg cr.

In some embodiments, the intestinal digestion function indicator comprises an indoleacetic acid (IAA) value and a phenylacetic acid (PAA) value; wherein clostridia metabolism indicator comprises a 4-hydroxyphenylacetic acid (4-HPAA) value, a p-cresol value, and a hydroxypropionic acid (HPHPA) value; wherein intestinal bacterial overgrowth indicator comprises a dihydroxyphenylpropionic acid (DHPPA) value, a hippurate value, a p-hydroxybenzoic acid (4-HBA) value, and a benzoate value; wherein the fungal overgrowth indicator comprises a citramalic acid value, a tartaric acid value and a tricarballylic acid value; wherein the oxalate metabolism indicator comprises a glycolate value, a glycerate value and a Oxalate value.

In some embodiments, the ideal value for the indoleacetic acid value is ≤37.6 μg/mg cr. and the reference value is ≤50.0 μg/mg cr., and the ideal value for the phenylacetic acid value is ≤1.70 μg/mg cr. and the reference value is ≤2.40 μg/mg cr.; wherein the ideal value for the 4-hydroxyphenylacetic acid value is ≤83.0 μg/mg cr. and the reference value is 99.8 μg/mg cr., the ideal value for the p-cresol value is ≤104 μg/mg cr. and the reference value is ≤146 μg/mg cr., and the ideal value for the hydroxypropionic acid value is ≤22.0 μg/mg cr. and the reference value is ≤40.2 μg/mg cr.; wherein the ideal value for the dihydroxyphenylpropionic acid value is ≤5.45 μg/mg cr. and the reference value is 6.20 μg/mg cr., the ideal value for the hippurate value is ≤405 μg/mg cr. and the reference value is ≤560 μg/mg cr., the ideal value for the p-hydroxybenzoic acid value is 5.90 μg/mg cr. and the reference value is ≤6.90 μg/mg cr., and the ideal value for the benzoate value is ≤1.21 μg/mg cr. and the reference value is 1.95 μg/mg cr.; wherein the ideal value for the citramalic acid value is ≤6.65 μg/mg cr. and the reference value is ≤7.70 μg/mg cr., the ideal value for the tartaric acid value is ≤7.80 μg/mg cr. and the reference value is ≤9.61 μg/mg cr., and the ideal value for the tricarballylic acid value is ≤2.31 μg/mg cr. and the reference value is ≤2.72 μg/mg cr.; wherein the ideal value for the glycolate value is ≤67.0 μg/mg cr. and the reference value is ≤83.0 μg/mg cr., the ideal value for the glycerate value is ≤5.22 μg/mg cr. and the reference value is ≤5.83 μg/mg cr., and the ideal value for the oxalate value is ≤19.1 μg/mg cr. and the reference value is ≤26.4 μg/mg cr.

In some embodiments, the application of organic acids in urine for health management further comprises a health management assessment unit, retrieving health-related promotion knowledge specific to the human body based on the indicators of the human body, in order to generate personalized health promotion recommendations.

### BRIEF DESCRIPTION OF THE DRAWINGS

The above and other objectives, features and other advantages of the present invention will be more clearly understood from the following detailed description taken in conjunction with the accompanying drawings, in which:

FIG. 1 illustrates a flowchart of detection procedures for an application of organic acid in urine for health management according to the present invention.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

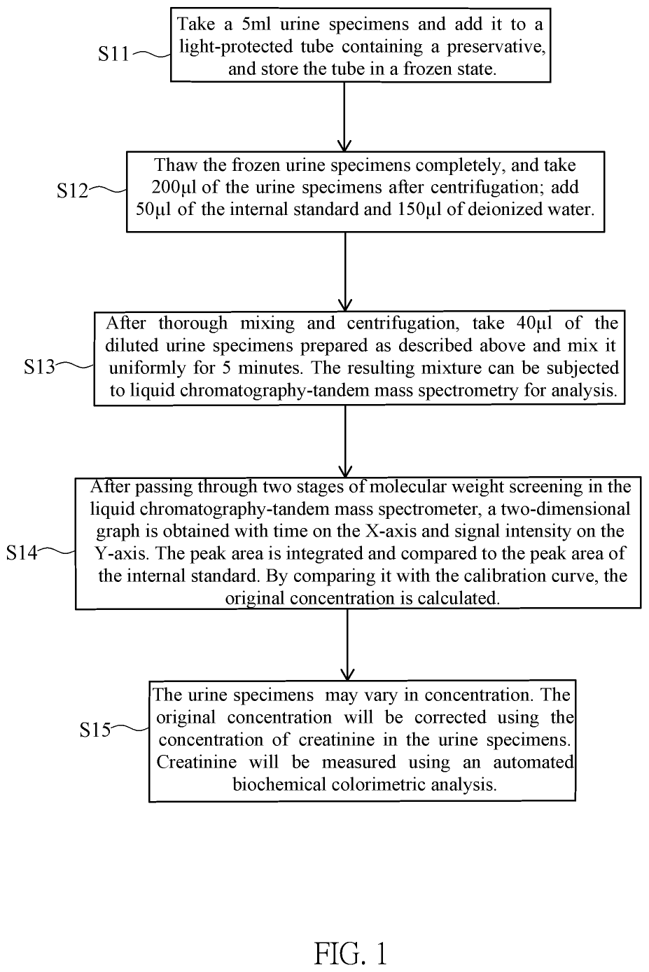

The following detailed description of the present application is taken in conjunction with the accompanying drawings, in which: Please refer to FIG. 1, FIG. 1 illustrates a flowchart of detection procedures for an application of organic acid in urine for health management according to the present invention.

The present invention provides an application of organic acids in urine for health management, the external testing of urine specimens from a human body was used, and a liquid chromatography tandem mass spectrometry was used to analyze the urine specimens to obtain multiple values of organic acids in the urine. As shown in FIG. 1, the detection procedures for the urine specimens are as follows. In step S11, the overnight urine specimens from a human body were collected, 5 ml of the urine specimens was taken and added to a light-protected tube containing a preservative, then stored in a frozen state. In step S12, before performing the examination, the frozen urine specimen was completely thawed during pre-processing. After centrifugation, 200 μl of the urine specimens was taken, and 50 μl of an internal standard and 150 μl of distilled water was added into the urine specimens. In step S13, after uniform mixing and centrifugation, 40 μl of the diluted urine specimens was taken and mixed thoroughly for 5 minutes. The urine specimens are then ready for analysis using liquid chromatography tandem mass spectrometry. In step S14, the liquid chromatography tandem mass spectrometer has been previously calibrated using pure standards to determine collision energy and fragmentation. The separation is performed using a C18 column with a gradient of water and acetonitrile. After passing through two stages of molecular weight screening, the sample enters the liquid chromatography tandem mass spectrometer. A two-dimensional plot is generated with time on the X-axis and signal intensity on the Y-axis. The peak areas are integrated and compared to the peak areas of internal standards. By comparing with the calibration curve, the original concentration is calculated. In addition, the urine specimens may exhibit variations in concentration, the original concentration is corrected using the concentration of creatinine in the urine specimens. Creatinine levels are measured using automated biochemical colorimetric analysis.

The organic acids detected in the urine specimens are classified as a first indicator, a second indicator, a third indicator, and a fourth indicator; wherein each urine organic acid has an ideal value and a reference value, when the organic acid values fall within the ideal value range, the human's health status is evaluated as healthy; when the organic acid values fall within the reference value range, the human's health status is evaluated as sub-healthy; and when the organic acid values fall outside the range of both the ideal value and reference value, the human's health status is evaluated as at risk of disease. The first indicator is selected from the group consisting of a fatty acid metabolism indicator, a carbohydrate metabolism indicator, and a mitochondrial energy generation indicator. The second indicator is selected from the group consisting of a vitamin B complex indicator, a urea cycle indicator, an oxidative damage DNA and lipid indicator, an antioxidant indicator, and a liver detoxification indicator. The third indicator is selected from the group consisting of a tryptophan metabolism indicator, a kynurenine metabolism indicator, a catecholamine metabolism indicator, and a catecholamine metabolism enzyme activity indicator. The fourth indicator is selected from the group consisting of an intestinal digestion function indicator, a clostridia metabolism indicator, an intestinal bacterial overgrowth indicator, a fungal overgrowth indicator, and an oxalate metabolism indicator.

### Fatty Acid Metabolism Indicator

During the metabolic process of fatty acids in the body, with the synergistic action of carnitine and vitamin B2, fatty acids are transported into the mitochondria for β-oxidation and converted into energy; wherein the fatty acid metabolism indicator comprises the adipate value of ≤5.15 μg/mg cr. as the ideal value and the reference value of ≤7.5 μg/mg cr., the suberate value of ≤4.58 μg/mg cr. as the ideal value and the reference value of ≤6.47 μg/mg cr., and the ethlmalonate value of ≤4.68 μg/mg cr. as the ideal value and the reference value of ≤6.12 μg/mg cr. Furthermore, the application of organic acids in urine for health management of the present invention further comprises a health management assessment unit. When there is insufficient carnitine or vitamin B2, inadequate acetyl CoA, or excessive insulin in the body, the β-oxidation of fatty acids is hindered, resulting in values of adipate, suberate, and ethylmalonate that are outside the normal range. Therefore, the health management assessment unit utilizes the fatty acid metabolism indicators to evaluate the human body's deficiency in carnitine and vitamin B2, and recommends supplementation of carnitine and vitamin B2.

### Carbohydrate Metabolism Indicator

In normal physiological conditions, the body primarily relies on carbohydrate metabolism as the main energy source. Glycolysis is the process of glucose energy metabolism, and it is also central to the dynamic balance of acetoacetate and lactate in the body; wherein the carbohydrate metabolism indicator comprises a pyruvate value, a lactate value and a beta-hydroxybutyric acid value. First, the ideal value for the pyruvate value is 2.06˜5.45 μg/mg cr. and the reference value is 0.23˜7.38 μg/mg cr.; wherein pyruvate is a normal metabolic product of carbohydrates. When the pyruvate value is low, it indicates a deficiency of cellular energy-generating substance. Furthermore, the ideal value for the lactate value is 7.51˜24.1 μg/mg cr. and the reference value is 2.8˜32.8 μg/mg cr. When the lactate value is low, it indicates insufficient materials for energy production derived from fatty acids, carbohydrates, and proteins. When the lactate value is high, it indicates cellular hypoxia or metabolic stress, which may result in muscle cramps and fatigue. Moreover, the ideal value for the beta-hydroxybutyric acid value is ≤2.27 μg/mg cr. and the reference value is ≤3.98 μg/mg cr. When cells are unable to obtain a steady supply of carbohydrates from the diet, cells will burn fat as an alternative energy source, leading to the production of ketone bodies

### Mitochondrial Energy Generation Indicator

By detecting the intermediate metabolites of the mitochondrial citric acid cycle, it is possible to evaluate the mitochondrial energy generation status and assess the deficiency of nutrients or proteins required for enzymes in the citric acid cycle; wherein the mitochondrial energy generation indicator comprises a citrate value, a cis-aconitate value, a isocitrate value, an alpha-ketoglutaric acid value, a succinate value, a fumarate value, a malate value, and a hydroxymethylglutaric acid value. First, the ideal value for the citrate value is 241˜575 μg/mg cr. and the reference value is 63˜748 μg/mg cr., the ideal value for the cis-aconitate value is 49˜83.6 μg/mg cr. and the reference value is 29.5˜103 μg/mg cr., and the ideal value for the isocitrate value is 58.3˜95.9 μg/mg cr. and the reference value is 38.8˜116 μg/mg cr.; wherein citrate, cis-aconitate and isocitrate are involved in energy generation and the process of toxic ammonia clearance. When the values of citrate, cis-aconitate and isocitrate are elevated, it may indicate poor liver detoxification capacity and imbalanced gut microbiota. When the values of citrate, cis-aconitate and isocitrate are low, it indicates indirectly an abnormality in upstream metabolites, and insufficient amino acids or poor digestion. The health management assessment unit will recommend supplementing with glutathione and amino acids for the human body. Furthermore, the ideal value for the alpha-ketoglutaric acid value is 5.5˜17.9 μg/mg cr. and the reference value is 0.9˜23.7 μg/mg cr.; wherein alpha-ketoglutaric acid requires vitamins B1, B2, B3, B5, and lipoic acid for its metabolism. When the value of α-ketoglutarate is elevated, it indicates a deficiency in vitamin B complex and lipoic acid, or accumulation of toxic metals. When the level of alpha-ketoglutaric acid is low, it indicates insufficient amino acids or digestive disorders; therefore, the health management assessment unit will recommend supplementing with B1, B2, B3, B5, lipoic acid, or amino acids. Moreover, the ideal value for the succinate value is ≤10.8 μg/mg cr. and the reference value is ≤18.7 μg/mg cr.; the ideal value for the fumarate value is ≤0.52 μg/mg cr. and the reference value is ≤0.79 μg/mg cr.; and the ideal value for the malate value is ≤1.34 μg/mg cr. and the reference value is ≤2.00 μg/mg cr. When the succinate value, the fumarate value, and the malate value are elevated, it indicates a possible deficiency of coenzyme Q10; therefore, the health management assessment unit will recommend supplementing with CoQ10 or amino acids. Additionally, the ideal value for hydroxymethylglutaric acid value is ≤5.96 μg/mg cr. and the reference value is ≤7.27 μg/mg cr. Hydroxymethylglutaric acid is used by cells to produce coenzyme Q10. When the hydroxymethylglutaric acid value is elevated, it indicates a possible deficiency of coenzyme Q10, and the health management assessment unit recommends supplementing with CoQ10 or amino acids.

### Vitamin B Complex Indicator

Vitamin B complex is an essential nutrient for cellular metabolism and numerous physiological responses; therefore, insufficient intake of vitamin B complex in the diet can affect mitochondrial function and increase the risk of chronic diseases. The vitamin B complex indicators comprise a α-ketoisovaleric acid value, a α-ketoisocaproic acid value, a α-keto-β-methylvaleric acid value, a xanthurenate value, a methylmalonic acid value, a N-Methylglutamic acid value and a β-Hydroxyisovaleric acid value. First, the α-ketoisovaleric acid value is ≤0.13 μg/mg cr. and the reference value is ≤0.2 μg/mg cr., the ideal value for the α-ketoisocaproic acid value is ≤0.63 μg/mg cr. and the reference value is ≤0.88 μg/mg cr., and the ideal value for the α-keto-β-methylvaleric acid value is ≤1.37 μg/mg cr. and the reference value is ≤2.13 μg/mg cr. B1, B2, B3, B5, and lipoic acid are coenzymes for the metabolic enzymes of α-ketoisovaleric acid, α-ketoisocaproic acid, and α-keto-β-methylvaleric acid. The deficiency of B1, B2, B3, B5, and lipoic acid impairs normal metabolism, resulting in elevated α-ketoisovaleric acid value, α-ketoisocaproic value, and α-keto-β-methylvaleric acid value. This deficiency may also be associated with toxic metals and high glucose levels in diabetes. Furthermore, the ideal value for the xanthurenate value is ≤0.49 μg/mg cr. and the reference value is ≤0.63 μg/mg cr. When the xanthurenate value is elevated, it indicates a deficiency of vitamin B6; therefore, the health management assessment unit will recommend supplementing with vitamin B6. In addition, the ideal value for the methylmalonic acid value is ≤2.27 μg/mg cr. and the reference value is ≤2.83 μg/mg cr. When the methylmalonic acid value is elevated, it indicates a deficiency of vitamin B12. Alternatively, the elevated methylmalonic acid value may also be related to low GSH affecting the conversion of vitamin B12; therefore, the health management assessment unit will recommend supplementing with vitamin B12. Moreover, the ideal value for the N-Methylglutamic acid value is ≤0.28 μg/mg cr. and the reference value is ≤0.38 μg/mg cr. When the N-Methylglutamic acid value is elevated, it indicates a deficiency of folic acid or metabolic abnormalities, which could be due to impaired methylation and/or elevated levels of homocysteine. In addition, the ideal value for the β-Hydroxyisovaleric acid value is ≤13.3 μg/mg cr. and the reference value is ≤17.0 μg/mg cr. When the β-Hydroxyisovaleric acid value is high, it indicates a deficiency of biotin; therefore, the health management assessment unit will recommend supplementing with biotin.

### Urea Cycle Indicator

When there is abnormal urea cycle metabolism, excessive ammonia in the body is converted into lactate. It may be associated with drug-induced liver toxicity, viral hepatitis, or congenital metabolic disorders. Excessive ammonia can affect the brain and lead to symptoms such as headaches, lack of concentration, and anxiety. The ideal value for the orotate value is ≤0.66 μg/mg cr. and the reference value is ≤0.90 μg/mg cr. If the orotate value is high, along with elevated values of citric acid, cis-aconitic acid, and isocitric acid, it indicates poor ammonia clearance; therefore, the health management assessment unit will recommend supplementing with arginine or N-acetylglutamine.

### Oxidant Damage DNA and Lipid Indicator

The byproducts of mitochondrial energy metabolism are free radicals. When the body's antioxidant system is insufficient to eliminate the free radicals, the body can react with cellular DNA, lipids, proteins, and other structures, leading to the generation of more oxidative substances and an increase in oxidative stress. Studies have found that high oxidative stress is associated with an increased risk of various diseases, such as cardiovascular, autoimmune diseases and cancer. The oxidant damage DNA indicator and lipid indicator comprises a deoxyguanosine value and a lipid peroxide value. First, the ideal value for the deoxyguanosine value is ≤4.78 μg/mg cr. and the reference value is ≤5.6 μg/mg cr.; wherein deoxyguanosine is a product of free radical attack on DNA in the body. The deoxyguanosine value can indicate the extent of oxidant damage to cellular DNA; therefore, the health management assessment unit recommends supplementing with antioxidant nutrients. Furthermore, the ideal value for the lipid peroxide value is ≤7.53 μg/mg cr. and the reference value is ≤9.8 μg/mg cr.; wherein lipid peroxides are the oxidative products which are generated when free radicals attack lipids in the body. Excessive lipid peroxides (LPO) can cause cellular damage and trigger immune-inflammatory response; therefore, the health management assessment unit recommends supplementing with antioxidant nutrients.

### Antioxidant Indicator

Glutathione (GSH) is one of the most potent antioxidants in the body. When the body is under high oxidative stress, the body compensates by increasing the synthesis of glutathione. The antioxidant indicator comprises an alpha-hydroxybutyric acid value and a pyroglutamate value. First, the ideal value for the alpha-hydroxybutyric acid value is ≤6.12 μg/mg cr. and the reference value is ≤7.37 μg/mg cr., alpha-hydroxybutyric acid is an indicator of a synthesis rate of liver glutathione and also an early indicator of insulin resistance and impaired glucose tolerance. Furthermore, the ideal value for the pyroglutamate value is 12˜22.1 μg/mg cr. and the reference value is 6.76˜27.3 μg/mg cr.; wherein pyroglutamate is a recycling indicator of renal and intestinal amino acid. When the pyroglutamate value is elevated, it indicates that the body has depleted glutathione (GSH) to prevent the loss of other amino acids. When the pyroglutamate value is low, it indicates inadequate intake of glutamate or deficiency of precursors, toxic burden, and exposure to oxidative stress; therefore, the health management assessment unit recommends supplementing glutathione.

### Liver Detoxification Indicator

When there is an excessive accumulation of toxins within cells, it can potentially impair mitochondrial function, such as metabolites of gut microbiota, prescription medications, pro-inflammatory foods, and so on; therefore, the load of the body's detoxification can be evaluated through liver detoxification index. The liver detoxification indicator comprises the glucarate value of ≤8.39 μg/mg cr. as the ideal value and the reference value of 11.0 μg/mg cr.; wherein glucarate is required for the liver detoxification process, making it a potential indicator of liver detoxification function. When the liver is under a greater detoxification load or when the second phase of liver detoxification is impaired, the liver detoxification indicator will increase; therefore, the health management assessment unit may recommend supplementing with calcium saccharate or antioxidant nutrients.

### Tryptophan Metabolism Indicator

Tryptophan is the precursor of serotonin and has the function of assisting intestinal mucosal regeneration and inhibiting potential pathogenic bacteria through IL-22. It also serves as a raw material for enterochromaffin cells to synthesize serotonin. When tryptophan is metabolized in the liver, it is converted to quinolinic acid in the presence of sufficient vitamin B6, which then enters the pathway for NAD synthesis. The tryptophan metabolism indicator comprises a tryptophan value, a 5-hydroxyindoleacetic acid value, a kynurenine value, a kynurenate value, a picolinate value and a quinolinate value. First, the ideal value for the tryptophan value is 6.74˜17.1 μg/mg cr. and the reference value is 5.39˜23.4 μg/mg cr., wherein when there is insufficient tryptophan in the intestines, it can affect gastrointestinal function and be associated with inflammatory bowel disease (IBD) and irritable bowel syndrome (IBS). When the values of tryptophan, phenylalanine, and tyrosine simultaneously increase in urine, it indicates a deficiency in tetrahydrobiopterin (BH4) and vitamin B6, and when the values is low, it may be related to inadequate protein intake or could be due to inflammation of the intestinal mucosa and alterations in the gut microbiota. Furthermore, the ideal value for 5-hydroxyindoleacetic acid value is 4.14˜8.10 μg/mg cr. and the reference value is 3.25˜10.1 μg/mg cr., if the 5-hydroxyindoleacetic acid (5-HIAA) value is abnormally elevated, it is recommended to rule out gastrointestinal neuroendocrine tumors. When the 5-hydroxyindoleacetic acid value is low, it indicates insufficient serotonin, and it may be related to a deficiency of precursor tryptophan, deficiency of co-factors BH4 or vitamin B6. Common symptoms include constipation, depression, fatigue, and lack of concentration; therefore, the health management assessment unit will recommend supplementing with vitamin B6 or tryptophan. Moreover, the ideal value for the kynurenine value is 0.34˜1.11 μg/mg cr. and the reference value is 0.27˜1.44 μg/mg cr. Tryptophan is metabolized into indoleamine by the TDO enzyme in the liver, and inflammatory will stimulate the IDO enzyme, which increases the synthesis of kynurenine. In addition, the ideal value for the kynurenate value is ≤2.84 μg/mg cr. and the reference value is ≤4.86 μg/mg cr., wherein kynurenate has neuroprotective effects and can act as an NMDA receptor antagonist. It possesses anti-inflammatory properties, protects nerves from oxidative stress, and reduces pain. When the kynurenate value is elevated, it may be due to insufficient expression of the KMO gene or enzyme damage. Studies suggests that an overactivation of a synthesis pathway of astrocytic and kynurenate (NMDA antagonist) is associated with schizophrenia. Also, the ideal value for the picolinate value is 0.12˜0.30 μg/mg cr. and the reference value is 0.072˜0.38 μg/mg cr., and the ideal value for the quinolinate value is ≤5.93 μg/mg cr.; wherein picolinate is one of the metabolites of quinolinic acid during the formation of NAD in the liver, and it activates Th1-related inflammatory cytokines in the immune system. Additionally, the ideal value for the quinolinate value is ≤5.93 μg/mg cr. and the reference value is ≤6.96 μg/mg cr. During inflammatory responses, macrophages stimulate microglia to synthesize quinolinic acid, which exhibits neurotoxicity in the brain and reduces mitochondrial activity.

### Kynurenine Metabolism Indicator

The metabolism of kynurenin is closely associated with inflammatory responses, the immune system, and neurological disorders. During inflammation, macrophages increase the synthesis of kynurenin through the action of IDO enzyme. Astrocytes in the brain convert kynurenate from kynurenine in the brain, which has neuroprotective effects and acts as an antagonist of NMDA receptors. It exhibits anti-inflammatory properties, protects neurons from oxidative stress, and reduces pain. The kynurenine metabolism indicator comprises a IDO enzyme activity value and a KMO enzyme activity value. First, the ideal value for the IDO enzyme activity value is 41.4˜90.4 μg/mg cr. and the reference value is 34.9˜104 μg/mg cr.; wherein the IDO enzyme activity value is increased by pro-inflammatory cytokines such as IFN-α, IFN-β, IFN-γ, TNF-α, IL-6, and PAF, prompting macrophages to synthesize kynurenine from tryptophan. Furthermore, the ideal value for the KMO enzyme activity value is 1.38˜3.22 μg/mg cr. and the reference value is 0.85˜4.46 μg/mg cr.; wherein IFN-γ stimulates microglia and macrophages to synthesize quinolinate through Th1 response. Quinolinate stimulates NMDA receptors, inhibits antioxidant enzyme activity, and reduces mitochondrial activity. Studies have found that the values of cortical kynurenate are elevated in the brains and cerebrospinal fluid of patients with schizophrenia, possibly due to decreased expression of the KMO gene or enzyme impairment. The health management assessment unit would recommend supplementation with SPMs, Theracurmin®, or Omega-3.

### Catecholamine Metabolism Indicator

The metabolites of dopamine, norepinephrine, and epinephrine can indicate the body's response to stress, primarily associated with internal emotional stress or exposure to environmental toxins. Whether an adequate supply of the precursor amino acids phenylalanine and tyrosine for catecholamine synthesis is available, or if their metabolism by gut microbiota affects the enzymatic activity of neurotransmitter metabolism. The catecholamine metabolism indicator comprises a ferulic acid value, a vanillin mandelic acid value and a dihydroxyphenylacetic acid value. First, the ideal value for the ferulic acid value is 2.81˜9.32 μg/mg cr. and the reference value is 1.99˜10.4 μg/mg cr.; wherein ferulic acid is the metabolic product of dopamine, a neurotransmitter, showing the neuronal response mediated by dopamine expression in the nervous system. When the ferulic acid value is elevated, it indicates being in a state of stress or excitement, leading to accelerated metabolism of dopamine. Furthermore, the ideal value for the vanillin mandelic acid value is 2.89˜5.33 μg/mg cr. and the reference value is 1.97˜5.93 μg/mg cr.; wherein the vanillin mandelic acid is a metabolite of norepinephrine and epinephrine, demonstrating the neuronal response mediated by the expression of norepinephrine and epinephrine in the nervous system. Moreover, the ideal value for the dihydroxyphenylacetic acid value is 0.72˜2.14 μg/mg cr. and the reference value is 0.54˜2.58 μg/mg cr.; wherein dihydroxyphenylacetic acid is another major metabolite of dopamine, indicating the normal synthesis of dopamine or the influence of exogenous environmental toxins on the synthesis of neurotransmitters. When the dihydroxyphenylacetic acid value is low, it may be associated with inadequate precursor amino acids for dopamine synthesis. The health management assessment unit will recommend the use of MAO inhibitors and supplementation with spore probiotics, or increasing intake of tyrosine.

Catecholamine metabolism enzyme activity indicator The metabolites of Clostridium butyricum, Fusaric acid (produced by fungi), disulfiram, food additive, such as aspartame, etc., can inhibit the enzyme activity of dopamine β-hydroxylase (DBH), while the COMT enzyme represents the methylation capability, which is related to the balance of neurotransmitters. The catecholamine metabolism enzyme activity indicator comprises a DBH enzyme activity value and a COMT enzyme activity value. First, the ideal value for the DBH enzyme activity value is 0.87˜2.11 μg/mg cr. and the reference value is 0.61˜2.84 μg/mg cr.; wherein the DBH enzyme activity value represents an activity of the enzyme dopamine beta hydroxylase (DBH) that converts dopamine into norepinephrine. When DBH enzyme activity value is elevated, it indicates insufficient DBH activity, typically due to the inhibition of the enzyme activity by metabolites of Clostridium butyricum, such as HPHPA, 4-cresol, and 4-HPAA. The health management assessment unit will recommend supplementing with spore probiotic. Furthermore, the ideal value for the COMT enzyme activity value is 2.75˜6.28 μg/mg cr. and the reference value is 1.48˜7.86 μg/mg cr.; wherein the COMT enzyme activity value is measured as a ratio of HVA to DOPAC, both of which are major metabolites of dopamine. The HVA/DOPAC ratio can indicate the activity of COMT enzyme and methylation capacity.

### Intestinal Digestion Function Indicator

The unabsorbed polyphenols, tyrosine, phenylalanine, or tryptophan in the intestine can be metabolized by bacteria, reflecting issues such as poor protein digestion, insufficient gastric acid, or imbalanced gut microbiota. The intestinal digestion function indicator comprises an indoleacetic acid value and a phenylacetic acid value. First, the ideal value for the indoleacetic acid value is ≤37.6 μg/mg cr. and the reference value is ≤50.0 μg/mg cr.; wherein indoleacetic acid is a metabolite resulting from the bacterial breakdown of unabsorbed tryptophan in the intestines, and it is associated with overgrowth of Clostridia bacteria in the gut. When the indoleacetic acid value is elevated, it indicates poor absorption of tryptophan, which may be associated with insufficient gastric acid, imbalanced gut microbiota, and overgrowth of Clostridia bacteria. The health management assessment unit will recommend supplementing with digestive enzymes or spore probiotics. Furthermore, the ideal value for the phenylacetic acid value is 1.70 μg/mg cr. and the reference value is ≤2.40 μg/mg cr.; wherein the phenylacetic acid value is elevated, it indicates poor absorption of tyrosine or phenylalanine, low stomach acid, or imbalanced gut microbiota. The health management assessment unit will recommend supplementing with digestive enzymes or spore probiotics.

### Clostridia Metabolism Indicator

The gut microbiota metabolites of tryptophan, tyrosine, and phenylalanine are particularly associated with overgrowth of Clostridia; wherein the clostridia metabolism indicator comprises a 4-hydroxyphenylacetic acid (4-HPAA) value, a p-cresol value, and a hydroxypropionic acid (HPHPA) value. First, the ideal value for the 4-hydroxyphenylacetic acid value is ≤83.0 g/mg cr. and the reference value is ≤99.8 μg/mg cr.; wherein 4-hydroxyphenylacetic acid is a metabolite resulting from the bacterial breakdown of unabsorbed tyrosine in the intestines, and it is associated with overgrowth of Clostridia bacteria in the gut. When the 4-hydroxyphenylacetic acid value is elevated, it is associated with infections caused by the intestinal parasite Giardia lamblia, overgrowth of anaerobic bacteria due to antibiotic misuse, excessive growth of Clostridia bacteria, abnormal proliferation of small intestinal bacteria, or small intestinal disorders. Furthermore, the ideal value for the p-cresol value is ≤104 μg/mg cr. and the reference value is ≤146 μg/mg cr.; wherein p-cresol is a metabolite resulting from the bacterial breakdown of unabsorbed phenylalanine in the intestines, and it is associated with overgrowth of Clostridia bacteria in the gut. When the p-cresol value is elevated, it indicates the overgrowth of the problematic Clostridium difficile bacteria in the intestines, whose secreted toxins can cause mild to moderate diarrhea and pseudomembranous colitis. Moreover, the ideal value for the hydroxypropionic acid value is ≤22.0 μg/mg cr. and the reference value is ≤40.2 μg/mg cr.; wherein hydroxypropionic acid is a metabolite resulting from the bacterial breakdown of unabsorbed phenylalanine in the intestines, and it is associated with overgrowth of Clostridia bacteria in the gut. When the hydroxypropionic acid value is elevated, it indicates an overgrowth of Clostridia and Clostridium botulinum bacteria, which secrete neurotoxins such as botulinum toxin. The associated symptoms include weakness in the limbs and chest muscles, visual impairment, fatigue, and speech difficulties; wherein the botulinum toxin can potentially originate from contaminated food. The health management assessment unit will recommend supplementing with spore probiotics.

### Intestinal Bacterial Overgrowth Indicator

Overgrowth of gut microbiota leads to the breakdown of incompletely absorbed amino acids, chlorogenic acids, and polyphenols in the intestines, resulting in an increase in metabolites. The use of antibiotics affects the balance of gut microbiota, while exposure to preservatives in cosmetics, fruit jams, and pie fillings increases the value of hydroxybenzoic acid esters (parabens) in the body. Similarly, consuming foods such as pickled vegetables, ham, and sausages that contain benzoic acid as a preservative can lead to an increase in intestinal bacterial overgrowth indicator. The intestinal bacterial overgrowth indicator comprises a dihydroxyphenylpropionic acid value, a hippurate value, a p-hydroxybenzoic acid (4-HBA) value, and a benzoate value. First, the ideal value for the dihydroxyphenylpropionic acid value is ≤5.45 μg/mg cr. and the reference value is ≤6.20 μg/mg cr.; wherein dihydroxyphenylpropionic acid is the partially absorbed form of phenylalanine, or the metabolite produced by bacterial breakdown of chlorogenic acid found in beverages, fruits, and vegetables. When the dihydroxyphenylpropionic acid value is elevated, it indicates overgrowth of Clostridium difficile, Lactobacillus, Bifidobacterium, and Escherichia coli. Furthermore, the ideal value for the hippurate value is ≤405 μg/mg cr. and the reference value is ≤560 μg/mg cr.; wherein hippuric acid is a metabolite formed by bacterial breakdown of unabsorbed phenylalanine in the intestines, and it is also a combination of benzoic acid and glycine. When there is an overgrowth of gut microbiota, bacteria will break down polyphenols, especially chlorogenic acid, found in beverages, vegetables, and fruits. As a result, the level of hippuric acid will increase. The health management assessment unit would recommend supplementing with spore probiotics. Additionally, the ideal value for the p-hydroxybenzoic acid value is ≤5.90 μg/mg cr. and the reference value is ≤6.90 μg/mg cr., wherein para-Hydroxybenzoic acid is a bacterial metabolite of tyrosine, and antibiotics can promote the growth of the bacteria. Excessive presence of para-hydroxybenzoic acid esters, due to the use of cosmetics, can result in elevated values of para-hydroxybenzoic acid. When para-hydroxybenzoic acid is elevated, it indicates an excess of preservatives such as para-hydroxybenzoic acid esters, resulting in imbalanced gut microbiota, lactose intolerance or gluten sensitivity, as well as enteritis or intestinal resection. The health management assessment unit recommends supplementing with spore probiotics, glycine, or vitamin B5. Moreover, the ideal value for the benzoate value is ≤1.21 μg/mg cr. and the reference value is 1.95 μg/mg cr.; wherein benzoic acid naturally occurs in fruits such as berries and is commonly found in pickled foods and canned meats. It can also originate from the preservative, benzoic acid. It is associated with conditions like small intestinal bacterial overgrowth (SIBO), diverticular fibrosis, and celiac disease. When the hippurate value is within normal range while the benzoic acid value is elevated, it may be related to impaired conjugation of glycine during the liver's phase II detoxification. The health management assessment unit recommends supplementation with spore probiotics, glycine, or vitamin B5.

### Fungal Overgrowth Indicator

Fungi refer to yeasts, Candida, or fusariums, and their overgrowth in the body can contribute to increased inflammatory responses, possibly associated with autoimmune diseases. The fungal overgrowth indicator comprises a citramalic acid value, a tartaric acid value and a tricarballylic acid value. First, the ideal value for the citramalic acid value is ≤6.65 μg/mg cr. and the reference value is ≤7.70 μg/mg cr.; wherein citric acid is a metabolite of yeast, anaerobic bacteria such as Propionibacterium acnes, or Clostridium pasteurianum. When the citric acid value is elevated, it indicates an overgrowth of yeast or anaerobic bacteria; therefore, the health management assessment unit may recommend supplementation with Brewer's yeast. Furthermore, the ideal value for the tartaric acid value is ≤7.80 μg/mg cr. and the reference value is ≤9.61 μg/mg cr.; wherein tartaric acid is a product of the enzymatic breakdown of hyaluronic acid by Candida albicans, and it is also present in foods that contain tartaric acid. The tartaric acid value is high, it indicates a high intake of foods rich in tartaric acid (such as fruits) or certain carbonated beverages and the use of tartaric acid in baked goods. Additionally, the ideal value for the tricarballylic acid value is ≤2.31 μg/mg cr. and the reference value is ≤2.72 μg/mg cr.; wherein propionic acid is a metabolic byproduct released by the gut of fumonisins produced by Fusarium, which tightly binds with minerals and affects mineral absorption.

### Oxalate Metabolism Indicator

Excessive oxalate is not only associated with kidney stones but also with the formation of oxalate crystals in bones, joints, blood vessels, thyroid, or even in the brain. The sharp structure of oxalate crystals can cause pain and damage to various tissues, increase inflammation response, and interfere with the normal functioning of organs. The accumulation of oxalate in brain and vascular tissues can result in oxidative damage. Additionally, the accumulation of mercury oxalate crystals in tissues may also be one of the causes of autism. The high correlation between the excessive growth of fungi and the metabolites of arabitol and oxalate suggests that fungal overgrowth is likely one of the main causes of elevated oxalate in the autistic population; wherein the oxalate metabolism indicators include values of ethylmalonate, glycolate, and oxalate. First, the ideal value for the glycolate value is ≤67.0 μg/mg cr. and the reference value is ≤83.0 μg/mg cr., in the genetic disorder known as Hyperoxaluria Type 1 and in cases of vitamin B6 deficiency, there is insufficient enzymatic activity of alanine glyoxylate aminotransferase, which results in the conversion of glyoxylate to glycolate via other enzymes. Elevated levels of glycolate may indicate primary hyperoxaluria or vitamin B6 deficiency. Further, the ideal value for the glycerate value is ≤5.22 μg/mg cr. and the reference value is ≤5.83 μg/mg cr., in the genetic disorder known as Hyperoxaluria Type 2, there is a deficiency of the GRHPR enzyme, leading to an increased conversion of glyoxylate to oxalate. If oxalate and glycolate levels are simultaneously elevated, it may indicate Hyperoxaluria Type 2. Furthermore, the ideal value for the oxalate value is 19.1 μg/mg cr. and the reference value is ≤26.4 μg/mg cr., excessive oxalate is not only associated with kidney stones but also with primary hyperoxaluria, autism, fibromyalgia, and female vulvar pain. The elevation of oxalate levels may originate from exogenous high-dose vitamin C, ethylene glycol (antifreeze) in the environment, metabolites of arabitol from overgrowth of molds such as Aspergillus or Penicillium, or excessive growth of Candida. This health management assessment unit will recommend supplementing with vitamin B6 or Brewer's yeast.

The foregoing description, for the purpose of explanation, has been described with reference to specific embodiments. However, the embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the disclosure to the precise forms disclosed. Modifications and variations are possible in view of the above teachings.

I claim:

1. An application of organic acids in urine for health management, using for the external testing of urine specimens from a human body, using a liquid chromatography tandem mass spectrometry to analyze the urine specimens to obtain multiple values of organic acids in the urine, and categorizing the organic acid values as a first indicator, a second indicator, a third indicator, and a fourth indicator; wherein each urine organic acid has an ideal value and a reference value, when the organic acid values fall within the ideal value range, the human's health status is evaluated as healthy; when the organic acid values fall within the reference value range, the human's health status is evaluated as sub-healthy; and when the organic acid values fall outside the range of both the ideal value and reference value, the human's health status is evaluated as at risk of disease;
wherein the first indicator is selected from the group consisting of a fatty acid metabolism indicator, a carbohydrate metabolism indicator, and a mitochondrial energy generation indicator;
wherein the second indicator is selected from the group consisting of a vitamin B complex indicator, a urea cycle indicator, an oxidative damage DNA and lipid indicator, an antioxidant indicator, and a liver detoxification indicator;
wherein the third indicator is selected from the group consisting of a tryptophan metabolism indicator, a kynurenine metabolism indicator, a catecholamine metabolism indicator, and a catecholamine metabolism enzyme activity indicator;
wherein the fourth indicator is selected from the group consisting of an intestinal digestion function indicator, a clostridia metabolism indicator, an intestinal bacterial overgrowth indicator, a fungal overgrowth indicator, and an oxalate metabolism indicator.

2. The application of organic acids in urine for health management mentioned in claim 1, wherein the fatty acid metabolism indicator comprises an adipate value, a suberate value and an ethlmalonate value; the carbohydrate metabolism indicator comprises a pyruvate value, a lactate value and a beta-hydroxybutyric acid (BHBA) value; and the mitochondrial energy generation indicator comprises a citrate value, a cis-aconitate value, a isocitrate value, an alpha-ketoglutaric acid (AKG) value, a succinate value, a fumarate value, a malate value, a hydroxymethylglutaric acid (HMG) value.

3. The application of organic acids in urine for health management mentioned in claim 2, wherein the ideal value for the adipate value is ≤5.15 μg/mg cr. and the reference value is ≤7.25 μg/mg cr., the ideal value for the suberate value is ≤4.58 μg/mg cr. and the reference value is ≤6.47 μg/mg cr., and the ideal value for the ethlmalonate value is ≤4.68 μg/mg cr. and the reference value is ≤6.12 μg/mg cr; wherein the ideal value for the pyruvate value is 2.06˜5.45 μg/mg cr. and the reference value is 0.23˜7.38 μg/mg cr.; the ideal value for the lactate value is 7.51˜24.1 μg/mg cr. and the reference value is 2.8˜32.8 μg/mg cr., and the ideal value for the beta-hydroxybutyric acid value is ≤2.27 μg/mg cr. and the reference value is ≤3.98 μg/mg cr.; wherein the ideal value for the citrate value is 241˜575 μg/mg cr. and the reference value is 63˜748 μg/mg cr., the ideal value for the cis-aconitate value is 49˜83.6 μg/mg cr. and the reference value is 29.5˜103 μg/mg cr., the ideal value for the isocitrate value is 58.3˜95.9 μg/mg cr. and the reference value is 38.8˜116 μg/mg cr., the ideal value for the alpha-ketoglutaric acid value is 5.5˜17.9 μg/mg cr. and the reference value is 0.9˜23.7 μg/mg cr., the ideal value for the succinate value is ≤10.8 μg/mg cr. and the reference value is ≤18.7 μg/mg cr., the ideal value for the fumarate value is ≤0.52 μg/mg cr. and the reference value is ≤0.79 μg/mg cr., the ideal value for the malate value is ≤1.34 μg/mg cr. and the reference value is ≤2.00 μg/mg cr., and the ideal value for hydroxymethylglutaric acid value is ≤5.96 μg/mg cr. and the reference value is ≤7.27 μg/mg cr.

4. The application of organic acids in urine for health management mentioned in claim 1, wherein the vitamin B complex indicator comprises a α-ketoisovaleric acid (AKIV) value, a α-ketoisocaproic acid (AKIC) value, a α-keto-β-methylvaleric acid (AKBM) value, a xanthurenate value, a methylmalonic acid (MMA) value, a N-Methylglutamic acid (FLGLH) value and a β-Hydroxyisovaleric acid (BHIVA) value; wherein the urea cycle indicator comprises an orotate value; wherein the oxidative damage DNA and lipid indicator comprises a deoxyguanosine (8-OHdG) value and a lipid peroxide (LPO) value; wherein the antioxidant indicator comprises an alpha-hydroxybutyric acid (AHBA) value and a pyroglutamate value; and wherein the liver detoxification indicator comprises a glucarate value.

5. The application of organic acids in urine for health management mentioned in claim 4, wherein the ideal value for the α-ketoisovaleric acid value is ≤0.13 μg/mg cr. and the reference value is ≤0.2 μg/mg cr., the ideal value for the α-ketoisocaproic acid value is ≤0.63 μg/mg cr. and the reference value is ≤0.88 μg/mg cr., the ideal value for the α-keto-β-methylvaleric acid value is ≤1.37 μg/mg cr. and the reference value is ≤2.13 μg/mg cr., the ideal value for the xanthurenate value is ≤0.49 μg/mg cr. and the reference value is ≤0.63 μg/mg cr., the ideal value for the methylmalonic acid value is ≤2.27 μg/mg cr. and the reference value is ≤2.83 μg/mg cr., the ideal value for the N-Methylglutamic acid value is ≤0.28 μg/mg cr. and the reference value is ≤0.38 μg/mg cr., and the ideal value for the β-Hydroxyisovaleric acid value is ≤13.3 μg/mg cr. and the reference value is ≤17.0 μg/mg cr.; wherein the ideal value for the orotate value is ≤0.66 μg/mg cr. and the reference value is ≤0.90 μg/mg cr.; wherein the ideal value for the deoxyguanosine value is ≤4.78 μg/mg cr. and the reference value is ≤5.6 μg/mg cr., and the ideal value for the lipid peroxide value is ≤7.53 μg/mg cr. and the reference value is ≤9.8 μg/mg cr.; wherein the ideal value for the alpha-hydroxybutyric acid value is ≤6.12 μg/mg cr. and the reference value is ≤7.37 μg/mg cr., and the ideal value for the pyroglutamate value is 12˜22.1 μg/mg cr. and the reference value is 6.76˜27.3 μg/mg cr.; and wherein the ideal value for the glucarate value is ≤8.39 μg/mg cr. and the reference value is ≤11.0 μg/mg cr.

6. The application of organic acids in urine for health management mentioned in claim 1, wherein the tryptophan metabolism indicator comprises a tryptophan value, a 5-hydroxyindoleacetic acid (5-HIAA) value, a kynurenine value, a kynurenate value, a picolinate value and an quinolinate value; wherein the kynurenine metabolism indicator comprises a IDO enzyme activity value and a KMO enzyme activity value; wherein the catecholamine metabolism indicator comprises a ferulic acid (HVA) value, a vanillin mandelic acid (VMA) value and a dihydroxyphenylacetic acid (DOPAC) value; and wherein the catecholamine metabolism enzyme activity indicator comprises a DBH enzyme activity value and a COMT enzyme activity value.

7. The application of organic acids in urine for health management mentioned in claim 6, wherein the ideal value for the tryptophan value is 6.74˜17.1 μg/mg cr. and the reference value is 5.39˜23.4 μg/mg cr., the ideal value for 5-hydroxyindoleacetic acid value is 4.14˜8.10 μg/mg cr. and the reference value is 3.25˜10.1 μg/mg cr., the ideal value for the kynurenine value is 0.34˜1.11 μg/mg cr. and the reference value is 0.27˜1.44 μg/mg cr., the ideal value for the kynurenate value is ≤2.84 μg/mg cr. and the reference value is ≤4.86 μg/mg cr., the ideal value for the picolinate value is 0.12˜0.30 μg/mg cr. and the reference value is 0.072˜0.38 μg/mg cr., and the ideal value for the quinolinate value is ≤5.93 μg/mg cr. and the reference value is ≤6.96 μg/mg cr.; wherein the ideal value for the IDO enzyme activity value is 41.4˜90.4 μg/mg cr. and the reference value is 34.9˜104 μg/mg cr., and the ideal value for the KMO enzyme activity value is 1.38˜3.22 μg/mg cr. and the reference value is 0.85˜4.46 μg/mg cr.; wherein the ideal value for the ferulic acid value is 2.81˜9.32 μg/mg cr. and the reference value is 1.99˜10.4 μg/mg cr., the ideal value for the vanillin mandelic acid value is 2.89˜5.33 μg/mg cr. and the reference value is 1.97˜5.93 μg/mg cr., and the ideal value for the dihydroxyphenylacetic acid value is 0.72˜2.14 μg/mg cr. and the reference value is 0.54˜2.58 μg/mg cr.; wherein the ideal value for the DBH enzyme activity value is 0.87˜2.11 μg/mg cr. and the reference value is 0.61˜2.84 μg/mg cr., and the ideal value for the COMT enzyme activity value is 2.75˜6.28 μg/mg cr. and the reference value is 1.48˜7.86 μg/mg cr.

8. The application of organic acids in urine for health management mentioned in claim 1, wherein the intestinal digestion function indicator comprises an indoleacetic acid (IAA) value and a phenylacetic acid (PAA) value; wherein clostridia metabolism indicator comprises a 4-hydroxyphenylacetic acid (4-HPAA) value, a p-cresol value, and a hydroxypropionic acid (HPHPA) value; wherein intestinal bacterial overgrowth indicator comprises a dihydroxyphenylpropionic acid (DHPPA) value, a hippurate value, a p-hydroxybenzoic acid (4-HBA) value, and a benzoate value; wherein the fungal overgrowth indicator comprises a citramalic acid value, a tartaric acid value and a tricarballylic acid value; wherein the oxalate metabolism indicator comprises a glycolate value, a glycerate value and a oxalate value.

9. The application of organic acids in urine for health management mentioned in claim 8, wherein the ideal value for the indoleacetic acid value is ≤37.6 μg/mg cr. and the reference value is ≤50.0 μg/mg cr., and the ideal value for the phenylacetic acid value is 1.70 μg/mg cr. and the reference value is ≤2.40 μg/mg cr.; wherein the ideal value for the 4-hydroxyphenylacetic acid value is ≤83.0 μg/mg cr. and the reference value is ≤99.8 μg/mg cr., the ideal value for the p-cresol value is ≤104 μg/mg cr. and the reference value is ≤146 μg/mg cr., and the ideal value for the hydroxypropionic acid value is ≤22.0 μg/mg cr. and the reference value is ≤40.2 μg/mg cr.; wherein the ideal value for the dihydroxyphenylpropionic acid value is ≤5.45 μg/mg cr. and the reference value is ≤6.20 μg/mg cr., the ideal value for the hippurate value is ≤405 μg/mg cr. and the reference value is ≤560 μg/mg cr., the ideal value for the p-hydroxybenzoic acid value is ≤5.90 μg/mg cr. and the reference value is ≤6.90 μg/mg cr., and the ideal value for the benzoate value is ≤1.21 μg/mg cr. and the reference value is 1.95 μg/mg cr.; wherein the ideal value for the citramalic acid value is ≤6.65 μg/mg cr. and the reference value is ≤7.70 μg/mg cr., the ideal value for the tartaric acid value is ≤7.80 μg/mg cr. and the reference value is ≤9.61 μg/mg cr., and the ideal value for the tricarballylic acid value is ≤2.31 μg/mg cr. and the reference value is ≤2.72 μg/mg cr.; wherein the ideal value for the glycolate value is ≤67.0 μg/mg cr. and the reference value is ≤83.0 μg/mg cr., the ideal value for the glycerate value is ≤5.22 μg/mg cr. and the reference value is ≤5.83 μg/mg cr., and the ideal value for the oxalate value is ≤19.1 μg/mg cr. and the reference value is ≤26.4 μg/mg cr.
The application of organic acids in urine for health management mentioned in claim 1, further comprising a health management assessment unit, retrieving health-related promotion knowledge specific to the human body based on the indicators of the human body, in order to generate personalized health promotion recommendations.
